(19) United States
(12) Patent Application Publication
(10) Pub. No.: US 2010/0303911 A1
(43) Pub. Date: Dec. 2, 2010

(54) Hydrogel systems

(76) Inventors: Heather Sheardown, Nobleton (CA); David Morrison, London (CA); Scott Fitzpatrick, Oshawa (CA); Lina Liu, Hamilton (CA)

Correspondence Address: VALENTINE A COTTRILL;SUSAN TANDAN, 50 QUEEN STREET NORTH, STE. 1020, P.O. BOX 2248, KITCHENER, ON, N2H6M2, CA

(21) Appl. No.: 12/457,079

(22) Filed: Jun. 1, 2009

(51) Int. Cl. A61K 9/10

(52) U.S. Cl. 424/486

(57) ABSTRACT

A method of in situ hydrogel polymerization is provided. The method comprises combining a backbone polymer with an in situ polymerizable group to form a prepolymer solution which is administered to a target site and polymerized at the target site on exposure to a polymerization-inducing stimulus.

### FIELD OF THE INVENTION

The present invention relates to therapeutically useful hydrogel systems.

### BACKGROUND OF THE INVENTION

Permanent vision loss associated with diseases affecting the retina, such as age-related macular degeneration (AMD), diabetic retinopathy (DR), retinitis pigmentosa and glaucoma, affect over 10 million people worldwide. Advances in stem and progenitor cell technology have demonstrated the potential to repopulate diseased retinal tissue with viable, functioning photoreceptor cells, which can potentially lead to the restoration of sight. Unfortunately, current cell delivery techniques have significant drawbacks. Bolus cell injections result in poor delivery efficiency, whereas degradable cell scaffolds require invasive surgical techniques for implantation, which can have deleterious effects on an already compromised eye.

In addition, lens replacement surgery has become the most common surgical procedure performed in Canada. As life expectancy increases as well as the average age of the population, it is expected that this procedure will become even more commonplace in the future. Artificial intraocular lenses (IOLs) have progressed since their initial development in the early 1950's from simple, rigid pieces of polymethyl methacrylate (PMMA) to more complex designs that are foldable and therefore capable of being directly injected into the lens capsule, thereby reducing the occurrence of potentially harmful complications. Unfortunately, despite their rapid growth, two major obstacles remain in the design of currently available IOLs. Firstly, researchers have been unsuccessful in designing a lens that is capable of accommodating a person's vision to enable the continuous change of focal points required to effectively mimic native vision. Thus, patients that undergo lens replacement surgery still must depend on post-operative corrective lenses to provide near vision. Furthermore, researchers have been unable to design a lens that effectively eliminates the occurrence of posterior capsule opacification (PCO), more commonly known as secondary cataracts. Currently approximately 30% of patients with IOLs develop PCO within five years of surgery and must undergo a subsequent procedure.

The most recent research into artificial lenses has revolved around designs that take advantage of the natural mechanism of accommodation to provide changes in focus. For example, the AT-45 Crystalens has been shown to provide good distance, intermediate and near visual performance to patients, and is a significant improvement over traditional monofocal lenses. The ICU accommodative lenses have been shown to provide improved near visual acuity in comparison to monofocal lenses. However, capsule bag performance and the incidence of PCO appear to be worse with accommodating IOLs than with standard IOLs. This is believed to be due to the thin design of these lenses which allows for the movement of residual epithelial cells to the posterior surface of the lens.

Hydrogels that are directly implanted into the lens capsule in their functional form provide an interesting alternative to the traditional approach of IOL designs. Previous work has shown that completely refilling the capsular bag with viscoelastic materials results in minimal PCO.

Given the foregoing, it would be desirable to further develop hydrogel technologies in order to provide systems and methodologies that address therapeutic needs.

### SUMMARY OF THE INVENTION

Methods of polymerizing hydrogels in situ are provided in which a prepolymer is prepared, administered to a desired target site and exposed to a stimulus that induces polymerization at the target site. The method advantageously provides a novel means of therapy, for example, in connection with device application, tissue engineering and drug delivery.

Thus, in one aspect of the present invention, a method of in situ hydrogel polymerization is provided comprising the steps of:
1) modifying a biocompatible backbone polymer with an in situ polymerizable group to form a prepolymer solution;
2) administering the prepolymer solution to a target site; and
3) exposing the prepolymer solution to a stimulus that induces polymerization of the solution at the target site.

In another aspect of the invention, a prepolymer solution is provided comprising a collagen backbone and an acrylamide polymerizing agent.

In another aspect of the invention, a kit is provided comprising a polymer backbone and a polymerizable group, and optionally comprising one or more of a crosslinking agent, a facilitating agent, a stability agent, a polymerization initiating agent and a utility-specific component.

In a further aspect of the invention, an article of manufacture comprising packaging material and a prepolymer solution comprising a backbone polymer and a polymerizable group, wherein the packaging material is labeled to indicate that the prepolymer solution is for use to be administered to a target site for in situ polymerization on exposure to a stimulus that induces polymerization at the target site.

These and other aspects of the invention are described by reference to the following figures.

### BRIEF DESCRIPTION OF THE FIGURES

FIG. 1 graphically compares the water content of methacrylated HA hydrogels;

FIG. 2 graphically compares the swelling ratio of methacrylated HA hydrogels;

FIG. 3 graphically illustrates the transmission spectrums for methacrylated hydrogels;

FIG. 4 illustrates the light transmittance through methacrylated HA hydrogels containing a hydrophilic UV absorbent;

FIG. 5 graphically illustrates the refractive index of methacrylated HA hydrogels of various molecular weights and pre-polymer weight percents;

FIG. 6 graphically illustrates the maximum strength of a variety of methacrylated HA hydrogels;

FIG. 7 illustrates the elastic modulus (kPa) of various methacrylated HA hydrogels;

FIG. 8 graphically illustrates the numbers of live cells present on the surface of methacrylated HA hydrogels after 24 and 48 hour incubation periods;

FIG. 9 is a schematic depicting a scaffold in which linear chains of amine terminated PNIPAAm have been grafted to carboxylic acid groups of a collagen backbone;

FIG. 10 graphically illustrates the phase transition analysis of different PNIPAAm-collagen based scaffolds;

FIG. 11 graphically illustrates the viability of RPE cells suspended in a PCol scaffold at densities of 10,000 and 100,000 cells per well after 4 and 14 days, respectively; and

FIG. 12 graphically illustrates the cell count A) and viabilities B) of RPE cells cultured in a PCol suspension for periods of 4 and 14 days.

### DETAILED DESCRIPTION OF THE INVENTION

A method of in situ hydrogel polymerization is provided. The method includes modifying a biocompatible backbone polymer with an in situ polymerizable group to form a prepolymer solution, administering the prepolymer solution to a target site; and exposing the prepolymer solution to a stimulus that induces polymerization of the solution at the target site.

The backbone polymer is selected from a variety of suitable polymeric materials that are biocompatible. The term “biocompatible” is used herein to encompass polymers, and other materials, that are non-toxic and otherwise suitable for use with living tissue, for example in vitro and in vivo, and thus, are appropriate for use in biomedical applications, including for example, use in contact lenses, pacemaker leads and intraocular lenses. Examples of biocompatible polymers that may be utilized in the present method include, but are not limited to hyaluronic acid (HA), also known as “hyaluronan” or “hyaluronate”, having a molecular weight in the range of about 6000 to 300,000 kDa, and preferably, a molecular weight in the range of about 6,000 to about 30,000 kDa, protein-based biopolymers such as collagen, chitosan, poly (vinyl alcohol), poly (N vinyl pyrolidone), alginate, silicone-containing polymers such as (poly (dimethyl siloxane polymers)), including copolymers of methacryloxy propyl tris (trimethylsiloxy) silane (TRIS), and analogous synthetic bipolymers.

The selected biocompatible backbone polymer is modified with a biocompatible in situ polymerizable group to form a polymerizable prepolymer solution. In situ polymerizable groups include groups that polymerize, either on their own or on addition of a polymerization initiating agent, in response to a particular stimulus, for example, in response to a change in temperature, or in response to light. Examples of in situ polymerizable groups suitable for use in the present method, include monomers that form polymers including, but not limited to, acrylic-based polymers such as polymethylmethacrylate, poly (hydroxyethyl methacrylate) (pHEMA), poly N-isopropyl acrylamide, polyacrylic acid; polyurethanes and polyurethane ureas; silicone polymers and acrylic-based polymers such as pHEMA comprising various amounts of TRIS varying from about 1% to 99% TRIS; other hydrogel polymers including polyvinyl alcohol and protein-based biopolymers such as collagen. The in situ polymerizable group may also be a polymer formed by the foregoing monomers.

To form the prepolymer solution, a solution of the backbone polymer may be combined with a selected in situ polymerizable group under conditions suitable to permit the covalent attachment (or crosslinking) of the in situ polymerizable group to the backbone polymer, and for a suitable period of time to permit this covalent attachment to occur, for example, a period of time in the range of 1-3 days. As one of skill in the art will appreciate, in order to facilitate the crosslinking reaction, the in situ polymerizable group may be modified to increase reactivity, for example, may be modified to contain a more reactive functional group, examples of which include, but are not limited to, an anhydride group, a sulfhydryl group and a methacrylate group. The amount of polymerizable group combined with the backbone polymer is not particularly restricted. Generally, the polymerizable group is combined with the backbone polymer such that reactive groups on the backbone are utilized. Preferably, the polymerizable group is combined with the backbone polymer in excess to utilize all the reactive groups on the backbone polymer, and thereby prevent undesirable crosslinking from occurring, e.g. crosslinking between backbone polymer structures.

In addition, the reaction may be conducted in the presence of additional agents that promote, induce or facilitate a crosslinking reaction. In this regard, a crosslinking agent may be utilized in the reaction, including for example, a photocrosslinking agent such as a riboflavin-based crosslinking agent, or a crosslinking agent such as the heterobifunctional crosslinking agent, succinimidyl-4-(N-maleimidomethyl)cyclohexane-1-carboxylate (SMCC), that contains amine and sulfhydryl reactive end-groups. The cross-linking agent may also be, for example, UV light or heat. A facilitating agent may also be utilized, such as a carbodiimide, alone or in conjunction with a stability agent, such as N-hydroxysulfosuccinimide (NHS) or 1-Hydroxybenzotriazole (HOBT), which functions to increase the stability of the active intermediate, and may also increase the yield of the crosslinking reaction.

It will be appreciated by one of skill in the art, that a prepolymer solution comprising an in situ polymerizable group that interacts with a backbone polymer, and does not necessarily form covalent linkages with the backbone polymer, may also be polymerized in situ in accordance with the invention. In this regard, a polymerizable group may be modified to incorporate a functional group that forms a strong electrostatic interaction with the backbone polymer to form a prepolymer solution. In one embodiment, a fibronectin-functionalized polymerizable acrylamide is reacted with collagen backbone to form a prepolymer solution that polymerizes on application of heat.

The prepolymer solution is suitable for administration to a target site at which polymerization of the solution will desirably be induced when the solution is exposed to a polymerization-inducing stimulus. In this regard, polymerization may be induced on exposure or application of heat. In one embodiment, the application of heat to a prepolymer solution, e.g. a collagen-acrylamide (e.g. N-isopropylacrylamide) prepolymer solution, results in polymerization of the solution. Preferably, the prepolymer solution polymerizes at physiological temperature, e.g. at normal body temperature in the range of about 36-37.5° C., to render it suitable and convenient for use in vivo, e.g. the prepolymer solution polymerizes on administration to an in vivo target site.

Alternatively, the prepolymer solution may be admixed with a polymerization initiating agent that is sensitive to a given stimulus, such as heat or light, for polymerization of the solution to occur. In one embodiment, a photo-initiator is added to the prepolymer solution to form a photo-sensitive prepolymer solution. Examples of suitable photo-initiators include, but are not limited to, 2,2-dimethoxy-2-phenylacetophenone (DMPA), benzophenone and IRGACURE™. Generally the amount of photo-initiator added to the prepolymer solution is an amount sufficient to cause polymerization of the prepolymer solution on exposure to UV radiation, for example, an amount in the range of about 0.5-2% by weight of the prepolymer solution. For in situ polymerization, the photo-sensitive prepolymer solution is injected into a target site and exposed to UV light. The wavelength of UV light used for the polymerization reaction will depend on the photo-initiator used. For the photo-initiator, DMPA, a wavelength of about 365 nm is used.

The concentration of polymer in the prepolymer solution and the volume of prepolymer solution administered to a target site will vary with the particular application or treatment for which the solution is being employed. Generally, the prepolymer solution will include a polymer concentration in the range of about 1-10% by weight. The volume of prepolymer solution administered to a target site will be selected based on the intended application. Thus, for use in lens replacement therapy, a volume of the appropriate prepolymer solution suitable to function as an intraocular lens will be administered to the target lens site. For use in the delivery of cells or a therapeutic agent, a volume of the prepolymer solution suitable to deliver the required dose of cells or therapeutic agent will be administered, as well as amount suitable to retain the cells and/or agent at the target site, as required.

The in situ-formed polymer may be altered depending on the utility and desired properties thereof by selecting the backbone polymer and polymerizable group that will combine to yield a polymer with the desired characteristics. In one embodiment, thus, the backbone polymer is a hyaluronate combined with a methacrylate polymerizable group such as methacrylic anhydride which yields a mesh-like crosslinked polymer on exposure to light which is suitable for ophthalmic uses, such as lens replacement therapy. In another embodiment, collagen is utilized as the backbone polymer and an acrylamide (e.g. N-isopropylacrylamide) is utilized as the polymerizable group to render a unique, biocompatible in situ-formed polymer that may be more fibrous in nature on application of heat. The properties of either of these polymers may be altered by changing the nature or molecular weight of the backbone polymer as well as the properties of the polymerizable group (e.g. density, molecular weight).

To enhance the utility of the in situ-formed polymer, utility-specific components may be added to the prepolymer solution to impart on the polymer a particular property or function that may be intrinsic at the target site, or required for the utility of the polymer at the target site. For example, for use of the in situ-formed polymer to replace an intraocular lens, a UV absorbing molecule may be incorporated into the prepolymer solution to impart on the polymer the ability to absorb UVA light similar to a native lens. In this regard, an example of a UV absorbing molecule includes 2-hydroxy-4-methoxybenzophenone-5-sulfonic acid. Other molecules appropriate for this utility are well-known to those of skill in the art.

In addition, the prepolymer solution may incorporate components to be delivered to a target site, including for example, cells such as healthy cells or undifferentiated stem cells to replace diseased cells at a site of disease. The prepolymer solution may also incorporate one or more therapeutic or prophylactic agents for delivery to a target site to treat or prevent the onset of disease.

The present method may be utilized to inject a prepolymer solution into a variety of target sites to provide an in situ-formed polymer, including for example, an intraocular site to replace intraocular lenses; sites at which delivery of healthy cells to replace diseased or unhealthy cells is required, e.g. into retinal tissue to deliver photoreceptor cells; sites at which delivery of therapeutic or prophylactic agents is required, e.g. the knee, the cardiovascular system.

In another aspect of the invention, a kit is provided comprising a polymer backbone and a polymerizable group. The kit may additionally comprise agents that promote, induce or facilitate the crosslinking reaction, including crosslinking agents, facilitating agents, stability agents, polymerization initiating agents, and utility-specific components (e.g. UV-absorbing molecule for application of the in situ-formed polymer in lens replacement). Alternatively, the kit may comprise a prepolymer solution in which the backbone polymer and the polymerizable group have already been combined, along with an appropriate polymerization initiating agent. As one of skill in the art will appreciate, the kit will comprise sufficient amounts of each component therein to yield at least an adequate amount of prepolymer solution for its intended utility.

In a further aspect of the invention, an article of manufacture is provided. The article of manufacture comprises packaging material and a prepolymer solution comprising a backbone polymer and a polymerizable group. The packaging material is labeled to indicate that the prepolymer solution is for use to be administered to a target site for in situ polymerization on exposure to a stimulus that induces polymerization.

Embodiments of the invention are described by reference to the following specific example which is not to be construed as limiting.

### Example 1

### Hyaluronic Acid-Methacrylate In Situ-Formed Polymer

### Methacrylation of HA

HA was purchased in molecular weights of 4.7, 35 and 132 kDa from Lifecore Biomedical (Chaska, Minn.). Methacrylic anhydride, sodium hydroxide (NaOH) and phosphate buffered saline (PBS) were obtained from Sigma Aldrich (Oakville, ON). All of the chemicals were used without further purification.

The methacrylation of hyaluronic acid has been adapted from the procedure of Smeds et al. J Biomed Mater Res 2001; 54:115-21, the relevant contents of which are incorporated herein by reference. Briefly, HA was dissolved at 1 wt % in deionized water (diH2O). This solution was then reacted with a 20-fold molar excess of methacrylic anhydride over a period of ˜48 hours. The solution was constantly stirred and kept in an ice bath to maintain a temperature of ˜4° C. The pH of the solution was also monitored and adjusted to ˜8 through the addition of 5 M sodium hydroxide. After the 48-hour reaction period, the solution was removed from the ice bath and dialyzed against diH₂O using a 3500 MWCO membrane for another 48 hours, lyophilized and stored frozen in its native powder form. ¹H-NMR analysis confirmed the methacrylation.

### Hydrogel Preparation

2,2-dimethoxy-2-phenylacetophenone (DMPA) was purchased from Sigma Aldrich (Oakville, ON). After the synthesis of methacrylated HA, the macromer was dissolved at various weight percentages in PBS prior to polymerization. These pre-polymer solutions were then mixed with a photoinitiator solution consisting of 33 wt % DMPA dissolved in methanol at a ratio of 1 wt % initiator to pre-polymer solution. This pre-polymer and initiator mixture was then injected into molds of various sizes using a pipette and placed into a UV oven (CureZone 2) where photopolymerization occurred upon exposure to 12.5 mW of 365 nm UV light over a period of ˜5 minutes.

### Hydrogel Characterization

### Water Content Studies

Hydrogel water content for the methacrylated HA samples was defined as the ratio of the weight of the polymer after complete dehydration to the weight of the polymer immediately after photopolymerization. Hydrogel disks were polymerized in ⅜″ diameter plastic molds, removed after polymerization and immediately weighed on a plastic weighing dish. Gels were then dehydrated on the weighing dish in a 70° C. oven for 120 minutes and subsequently weighed again.

### Swelling Ratio

The swelling ratio of the hydrogels was defined as the ratio of the weight of the hydrogels after they were swollen in PBS for 24 hours at 37° C. to the weight of the hydrogels after being fully dehydrated. Hydrogel disks were polymerized in ⅜″ diameter plastic molds, removed after polymerization and dehydrated in a weighting dish at 70° C. for 120 minutes. After each sample was weighed, the samples were removed from the weighing dish and placed in 48-well plates where 1 mL diH₂O was added. The 48-well plates were placed in an incubator for 24 hours.

The average mesh size and the effective crosslinking density of the hydrogels were also calculated using established techniques.

### Degradation

Hyaluronidase was purchased from Sigma Aldrich (Oakville, ON). For degradation analysis, polymer disks of ⅜″ diameter were degraded in solutions containing either 10 or 100 U hyaluronidase per mL of PBS. Samples were checked every 24 hours and the hyaluronidase solution was replaced every 48 hours throughout the study. Samples were kept at 37° C. for the entire experimental duration period. The time for complete degradation of the hydrogel disks was determined to be the point at which no piece of the hydrogel was visible within the plate well after the hyaluronidase solution had been removed.

### Optical Transparency

Light transmittance through the hydrogels was measured using a Beckman Coulter DU-640 spectrophotometer at a scan speed of 600 nm/min and an interval of 50 seconds. Light ranging in wavelength from 250 to 1000 nm was shone through ⅜″ methacrylated HA hydrogel disks and the amount of light transmitted was quantified.

In an attempt to mimic the transmission curve of the human lens, the pre-polymer form of methacrylated HA was mixed with 2-hydroxy-4-methoxybenzophenone-5-sulfonic acid (Sigma Aldrich, Oakville ON), a common UV absorbing molecule, at various concentrations and polymerized into hydrogels that were measured under the same conditions. It was determined that for complete dissolution of a UV absorbent solution, 10 wt % of 2-hydroxy-4-methoxybenzophenone-5-sulfonic acid was dissolved in diH₂O and then mixed with the methacrylated HA prepolymer solution.

### Refractive Index

The refractive index of methacrylated HA hydrogels was measured using an Atago model PAL-RI handheld digital refractometer. Previous studies have shown that this refractometer has been effective in measuring the refractive index of hydrogels. Hydrogel disks of ¼″ diameter were polymerized and placed onto the prism light sensor of the refractometer and proper coverage of the sensor was ensured to make certain that the measurements were accurate. Measurements were made in bright and dark ambient light conditions to verify the accuracy and repeatability of the instrument.

### Tensile Strength Testing

The tensile and compression strengths of the various hydrogel samples were measured at room temperature using an Instron Series XI Automated Material Testing System with a 50 N load. Samples with dimensions of 5 cm by 2 cm were cut from hydrogel samples polymerized in a custom mold and then cut into individual pieces. The custom mold consisted of a glass plate with glass microscope slides that acted as spacers and a petri dish as a cover piece. All surfaces were covered with parafilm as it was determined that this reduced the tendency of the hydrogel to stick to the surface of the mold and facilitated the removal of the hydrogel after photopolymerization. Care was taken to ensure that the hydrogel was not placed in the UV oven for too long as the parafilm would begin to melt after durations of greater than 5 minutes.

After samples were cut from the hydrogel sheet produced, they were loaded and tensile stress and strain were measured by the Instron mechanical tester at a crosshead speed of 10 mm/min. Elastic modulus and maximum tensile strength were reported.

### Cell Adhesion Studies

All materials were obtained from Invitrogen, Oakville, ON unless otherwise stated. To determine the interaction between the methacrylated HA hydrogels and cells present in the lens capsule, FHL-124 cells (a human lens epithelial cell line), were seeded onto the surface of the hydrogels and then imaged at various time points. Cells were only manipulated under sterile culture conditions and were maintained in a 95% air/5% CO₂ humidified incubator at 37° C. FHL-124 cells were maintained in 10% fetal bovine serum (FBS) in minimum essential medium (MEM). Pen-strep and gentamicin were also added to the media to minimize bacterial contamination. Confluent dishes of FHL-124 cells were passaged every 3-4 days. Only cells that had been passaged more than 10 and less than 15 times were used for cell seeding experiments in an attempt to maintain cell attachment consistency since it has been previously shown that the phenotype of these cells is altered with increased passage numbers.

Hydrogel disks were polymerized in the bottom of 96-well plates to ensure they were fixed and cells would not be able to migrate to their underside. The hydrogel surfaces were then sterilized with ethanol. After removing the ethanol from the surface, surfaces were rinsed with sterile PBS and FHL-124 cells were seeded onto the surfaces of each hydrogel using a bubble drop technique where a small bubble of the cell suspension was placed on top of the material surface and the cells were allowed to settle to the surface over a controlled time period. Cells were seeded at a density of 15,000 cells/cm² and immediately incubated for 120 minutes to facilitate cell attachment. After 120 minutes of incubation, 1 mL of media was added to each well and the cells were returned to the incubator for specified time periods. Cell viability was assessed using calcein AM and ethidium bromide.

### Results and Discussion

### Methacrylation of HA

The methacrylation reaction occurred on ice over a period of 48 hours. The reaction was confirmed using ¹H-NMR for HA molecular weights of 4.7 kDa, 35 kDa and 132 kDa. HA methacrylation was confirmed by the presence of methylene resonance peaks at ˜5.8 and 6.2 ppm.

### Hydrogel Preparation

Methacrylated HA of various molecular weights and weight percentages was investigated. The prepolymer concentrations investigated were chosen as the highest concentrations of methacrylated HA that had a low enough viscosity that they could be easily pipetted into the ⅜″ molds that were used for many of the characterization techniques. For example, the maximum weight percent that was investigated for 132 kDa HA was 5 wt %, but hydrogels of 10 wt % could be fabricated from 35 kDa HA.

During photopolymerization of the hydrogels, the prepolymer solution underwent free radical polymerization in the presence of UV light and an initiator. This reaction resulted in the formation of a covalent bond between two methacrylate end groups previously conjugated to the HA backbone.

### Hydrogel Characterization

### Water Content Studies

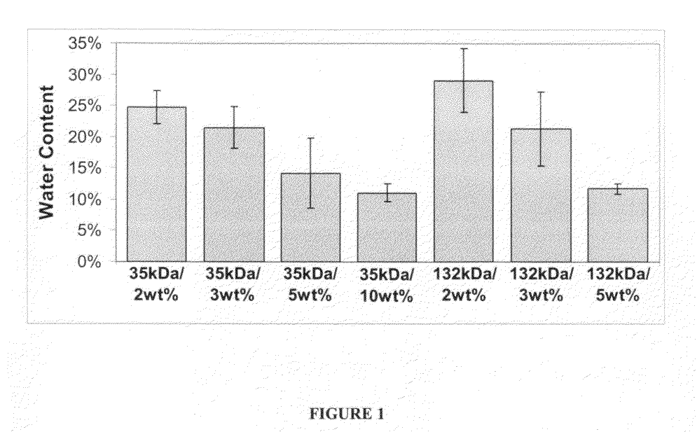

Water content of the methacrylated HA hydrogels was defined as the ratio of the weight of the polymer after complete dehydration to the weight of the polymer immediately after photopolymerization. FIG. 1 shows that as the molecular weight of the hydrogels increase the water content decreases correspondingly but that some control over the properties of the gels could be obtained by altering concentrations and molecular weights.

There is a similar response to increasing the weight percent of the pre-polymer solution. There are significant differences between the highest and lowest pre-polymer weight percents tested for each molecular weight. This result suggests that as the molecular weight and the weight percent of the pre-polymer solution increases, the degree of methacrylation of the HA-backbone remains consistent and the hydrogels get stronger due to an increase in the amount of crosslinking present within the hydrogel. This observation was also verified by using Flory-Rehner calculations to determine the mesh size and effective crosslink density values shown in Table 1.

TABLE 1

| | Average Mesh Size | Effective Crosslink |
|---|---|---|
| | (nm) | Density (×10⁶ mol/cm³) |
| 35 kDa/2 wt % | 676 | 1.364 |
| 35 kDa/3 wt % | 330 | 4.024 |
| 35 kDa/5 wt % | 202 | 7.780 |
| 35 kDa/10 wt % | 158 | 11.24 |
| 132 kDa/2 wt % | 399 | 2.936 |
| 132 kDa/3 wt % | 207 | 7.494 |
| 132 kDa/5 wt % | 167 | 10.27 |

The decreasing mesh size with molecular weight and weight percent as well as increasing effective crosslink density both indicate that increasing crosslinking occurs between hydrogels of higher molecular weight and weight percent. Because approximations regarding the root-mean-square distance between crosslinks were made in these calculations, these values are considered to be approximate. However, they provide good insight into the crosslinking properties of the various hydrogels.

### Swelling Ratio

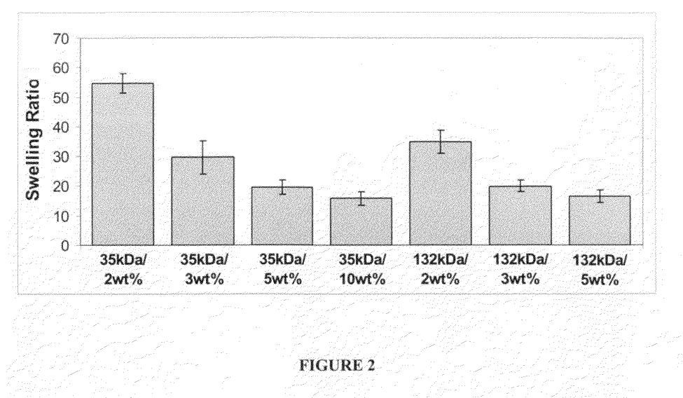

The swelling ratio of the hydrogels was defined as the ratio of the weight of the hydrogels after they were swollen in phosphate buffered saline (PBS) for 24 hours to the weight of the hydrogels after being fully dehydrated. As expected, a decrease in the swelling ratio is seen with an increase in the concentration of methacrylated HA in the prepolymer solutions (FIG. 2). For example, the swelling ratio is 54 for networks constructed of 2 wt % of the 35 kDa macromer but decreases to 15 when the macromer concentration is increased to 10 wt %. The same trend is seen for the 132 kDa methacrylated HA. For both molecular weights, there is a statistically significant (p>0.05) decrease in swelling ratio between the highest and the lowest methacrylated HA concentrations measured.

### Degradation

The overall time (measured in days) for complete degradation of the hydrogels of various molecular weights and prepolymer weight percentages in solutions of 10 and 100 U hyaluronidase/ml of PBS is shown in Table 2.

TABLE 2

| Hyaluronidase | 35 kDa | | | | 132 kDa | | |
|---|---|---|---|---|---|---|---|
| Concentration | 2 | | | 10 | 2 | | 5 |
| (U/mL PBS) | wt % | 3 wt % | 5 wt % | wt % | wt % | 3 wt % | wt % |
| 10 | 1 | 3 | 8 | 19 | 1 | 1 | 11 |
| 100 | 1 | 2 | 5 | 11 | 1 | 1 | 4 |

Hyaluronidase degrades the methacrylated HA hydrogels by cleaving the internal β-N-acetyl-D-glucosaminidic linkages, which results in fragments of N-acetylglucosamine at the reducing terminus and glucuronic acid at the non-reducing end. It was observed that the swollen networks decreased in size throughout their exposure to hyaluronidase during the degradation period. It appears that there is a good correlation between degradation time for each hydrogel and crosslinking density. For example, increasing the concentration of methacrylated HA resulted in extended times for complete degradation. This could potentially be due to an increase in gel surface erosion due to the restriction of the enzyme's ability to diffuse into the gel or an attraction between the positively charged groups produced during degradation and the negatively charged carboxylic acid groups of the HA. These results are only intended to provide insight into relative degradation times and do not represent actual times for degradation in the in vivo environment, as the local enzyme concentration varies greatly in different in vivo regions, such as different regions of the eye. Specifically, the measurements are intended to provide insight into alternate mechanisms of lens removal after implantation. Although there is hyaluronidase present naturally within the vitreous humour of the eye, given that the lens capsule is the thickest basement membrane in the human body, diffusion of the enzyme through this layer is expected to be negligible.

### Optical Transparency

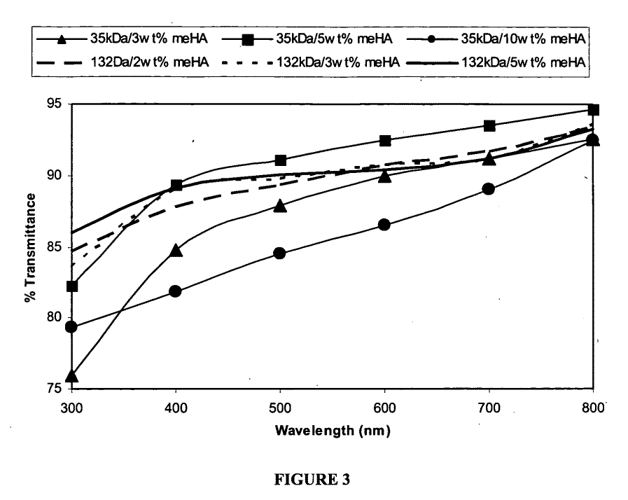

Light transmittance through the hydrogels was measured using a Beckman Coulter spectrophotometer. As shown in FIG. 3, methacrylated HA hydrogels in their native form allowed the transmittance of between 80 and 95% of light within the visual spectrum. Although there is no visible pattern or trend regarding the increase or decrease of the molecular weight or weight percent of methacrylated HA within the hydrogels, all six combinations measured permitted transmittances above 80% within the visual spectrum. According to the literature, these values are sufficient in mimicking the function of the human eye. These values are indicative of lens clarity.

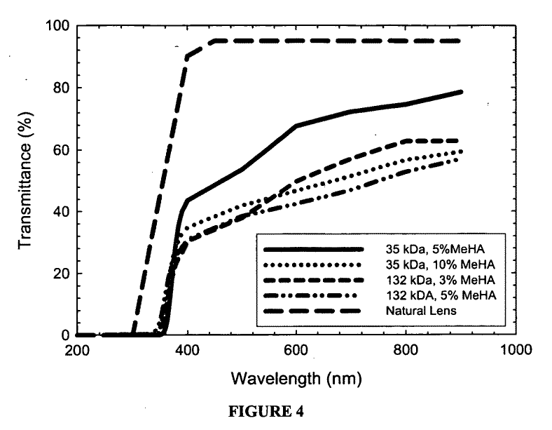

The native lens is responsible for the absorption of UVA light from the environment in order to protect the underlying retina. As such, a hydrophilic UV absorbing molecule was incorporated into the methacrylated HA precursor solution in an attempt to mimic this in vivo function. The pre-polymer form of methacrylated HA was mixed with a 10 wt % solution of 2-hydroxy-4-methoxybenzophenone-5-sulfonic acid, which acts by using the UV light as an energy source to initiate a conformation change in the 2-hydroxy-4-methoxybenzophenone-5-sulfonic acid molecule that is very unstable. Almost immediately after the formation of this unstable radical, it reverts back to its native form and gives off the energy acquired from the UV light as heat. The transmission curve shown in FIG. 4 shows that all UVB light and most UVA light is absorbed by the methacrylated HA hydrogels. The transmission spectrum of the modified methacrylated HA hydrogels is similar to that of an adolescent child, e.g. the transmission of visible light through the lens is approximately 70% of what is present in the surrounding environment.

### Refractive Index

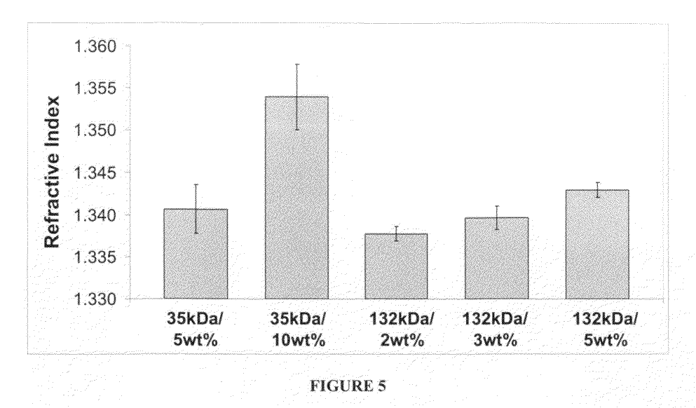

The refractive index of the methacrylated HA hydrogels is also a characteristic of the hydrogel to be identified in determining the potential ophthalmic applications of the hydrogel. Refractive index is the amount light waves change direction as they move through the medium. FIG. 5 shows the results of refractive index measurements for methacrylated HA hydrogels of two molecular weights. As expected, the refractive index of the hydrogels increased with increasing pre-polymer weight percent. This occurs as the HA molecules within the hydrogel act to reflect light waves at greater angles than water, of which the rest of the material consists.

### Tensile Strength Testing

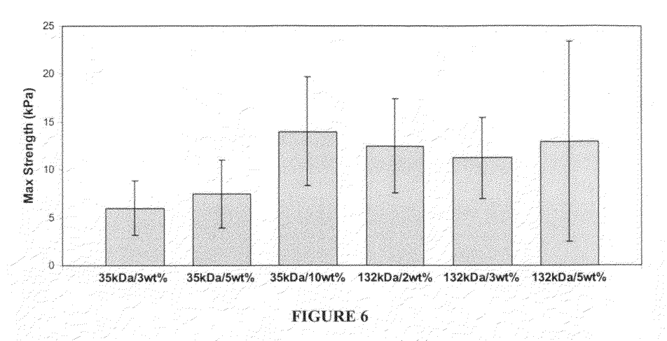

The tensile properties of the methacrylated HA hydrogels were measured by Instron mechanical testing. FIG. 6 summarizes the results for the maximum strength of various hydrogels before failure. Although there is no significant difference between the different molecular weights and weight percentages, it does appear as though the 132 kDa HA hydrogels are slightly stronger than the 35 kDa HA hydrogels and that hydrogels of higher weight percents are also stronger. This was the expected result as increasing the molecular weight of the polymer means that there are longer chain lengths and more entanglements within the polymer, which increase its tensile strength. Currently, no device has been developed that is capable of measuring forces exerted on the lens during accommodation in vivo; however, the external force acting on the human eye lens during accommodation using finite element modelling has been estimated to be 0.08 N. If the area of the lens is conservatively approximated to be 400 mm (the product of 10 mm diameter and 4 mm axial length), this equates to a force of 0.2 kPa, which the methacrylated HA greatly exceeds.

### Elastic Modulus/Flexibility

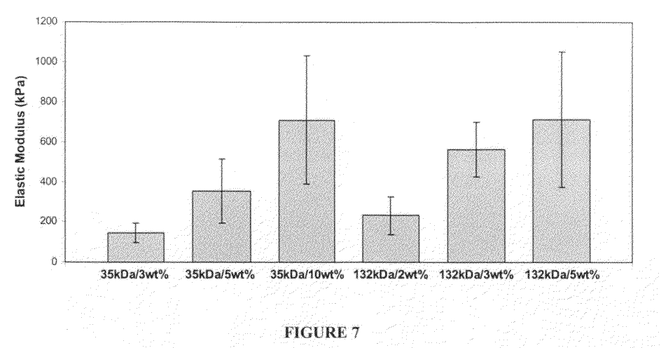

The elastic modulus (or Young's modulus) of a material gives insight into its stiffness and flexibility. FIG. 7 summarizes the results of measuring elastic modulus as the molecular weight and weight percent of the methacrylated HA hydrogels were varied. As the molecular weight and weight percent of the HA hydrogels increased, the elastic modulus increased correspondingly. In terms of applications as artificial IOLs, the elastic constant of the lens at birth is approximately 0.75 kPa and increases to 3 kPa at the age of 65. Therefore the methacrylated HA hydrogel elastic modulus greatly exceeds the limits of the human eye.

### Cell Adhesion Studies

Upon extraction of a cataractous lens and implantation of an IOL into the capsular bag, a breakdown of the blood-aqueous barrier occurs with immediate release of proteins and cells into the anterior chamber of the eye. Numerous biological interactions to the foreign IOL material occur, including non-specific protein deposition and complement activation, subsequent inflammation, foreign-body response, and cellular adhesion, transformation, migration and proliferation. To ascertain the cellular interactions between methacrylated HA hydrogels and lens epithelial cells, preliminary studies were performed where a human lens epithelial cell line, FHL-124 cells, were seeded onto the surface of the methacrylated HA hydrogels and then imaged under fluorescence.

A key characteristic in determining FHL-124 cell satisfaction is cell morphology. Lens epithelial cells must attach and spread out on their substrate before growth can occur and for proliferation to take place. When the cells retain a rounded shape and do not adhere, or adhere very loosely to the surface, they are not prone to growth and proliferation. In the present work, the seeded FHL-124 cells were shown to have a small round shape on the methacrylated HA hydrogels indicating that they are not likely to proliferate in the methacrylated HA hydrogel environment.

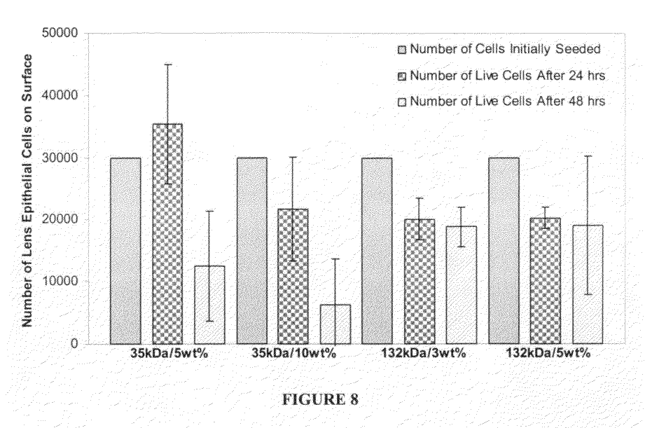

FIG. 8 shows that there is a noticeable reduction in the number of live cells over the 48-hour period for most hydrogel weight percents and molecular weights. This reduction is a clear indication of cell death. This response may be attributed to the hydrophilic nature of the methacrylated HA hydrogels. Since these cells require adequate adhesion to their surface in order to effectively lay down an extracellular matrix to promote growth; it appears as if there is little adhesion occurring. Lack of adhesion of these cells to the hydrophilic HA surface, thus, evidences the utility of the present hydrogel as an IOL with a reduction of interaction with the ocular environment.

### CONCLUSION

In this work HA was functionalized with methacrylate groups and formed into hydrogels through photopolymerization. These hydrogels were then characterized in terms of their potential for lens filling applications as IOLs. The results suggest that these types of HA-based hydrogels have tremendous potential as injectable IOL materials. Specifically, these hydrogel materials are capable of being molded into any shape and have viscoelastic properties that make them conducive to lens refilling. The resultant gels show similar optical transparency to the natural lens and when mixed with an appropriate UV-absorbing molecule, the transmittance spectrum within the visual range is similar to that of an adolescent child. It has also been shown that the refractive index of the HA-based hydrogel is close to the natural lens. Furthermore, lens epithelial cells appear to resist attachment to these HA hydrogels, thus suggesting their potential for reducing PCO in cataract patients.

### Example 2

### Collagen-PNIPAAm in Situ-Formed Polymer

### Synthesis of Amine Terminated PNIPAAm

N-isopropylacrylamide (NIPAAm, Sigma Aldrich) was purified by recrystallization from a toluene/hexane mixture. Amine terminated PNIPAAm was synthesized from NIPAAm via free radical polymerization using N,N′-Azobisisobutyronitrile (AIBN, Sigma) as an initiator and cysteamine hydrodrochloride (AESH, Sigma) as a chain transfer agent. NIPAAm, (88.37 mmol) and AESH (3.68 mmol) were dissolved in 20 ml DMF. Dry nitrogen was bubbled through the reaction mixture for thirty minutes prior to the addition of AIBN, previously recrystallized from methanol (1.22 mmol). Polymerization was allowed to proceed for 7 hours at 70° C. The polymerized product was precipitated into an excess of diethyl ether, where it was collected by decanting and purified by repeatedly precipitating and dissolving in water. The product was then dialyzed using dialysis tubing having MWCO 5,000 for three days, freeze-dried and stored at −20° C.

### Synthesis of PNIPAAm-Grafted-Collagen

### 1) Synthesis via EDC/NHS Chemistry

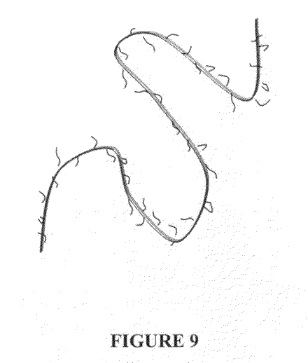

Bovine type I collagen was provided by Allergan. 1-Ethyl-(3-3-dimethylaminopropyl) carbodimiide hydrochloride (EDC, Sigma)/N-hydroxysuccinimide (NHS, Sigma) chemistry was used to graft linear chains of PNIPAAm onto a collagen backbone as illustrated in FIG. 9. EDC/NHS chemistry was used to generate covalent linkages between the carboxylic acid groups of aspartic acid (Asp) and glutamic acid (Glu) residues present in collagen with the amine functionalized end groups of the synthesized PNIPAAm. Briefly, 1 ml collagen (66 mg/ml) was acidified by thoroughly mixing with 100 μL HCl (1N). Amine terminated PNIPAAm (660 mg) was dissolved in PBS (5 ml, pH 7.2) and was added to the mixture. The pH was adjusted to 6.5 with HCl. A 600 μl solution of EDC and NHS crosslinkers (45 mg and 25.8 mg) dissolved in PBS was added to the mixture, which was de-gassed and allowed to react at room temperature for 24 hours. The mixture was then dialyzed for three days at 4° C. using dialysis tubing having MWCO 50,000 to remove any EDC, NHS and un-grafted PNIPAAm. The final product, linear chains of PNIPAAm grafted along the length of a collagen backbone, designated PCol, was freeze dried and stored at −20° C.

### 2) Synthesis via UV Photocrosslinking

Grafting of amine terminated PNIPAAm onto a collagen backbone was also achieved via UV photocrosslinking. The resulting copolymer was designated UV PCol. A riboflavin-based crosslinker was used to generate covalent linkages between terminal amine groups of PNIPAAm and carboxylic acid side chains present in collagen. The riboflavin photocrosslinker was provided by PriaVision. Collagen was acidified to pH 5.5 using 1N HCl. PNIPAAm was dissolved in PBS and mixed with collagen in a 2:1 (w/w) ratio. The UV crosslinker was added to the mixture in a 1:20 crosslinker to collagen (v/v) ratio. The mixture was then placed in a UV oven for 15 minutes, 365 nm, 12.5 W/cm².

Fibronectin Functionalized PNIPAAm Combined with Collagen

Succinimidyl-4-(N-maleimidomethyl)cyclohexane-1-carboxylate (SMCC), a heterobifunctional crosslinker that contains amine and sulfhydryl reactive end-groups, was purchased from Pierce Chemical. 10 mg of amine terminated PNIPAAm and 2 mg SMCC were dissolved in 1 ml PBS, pH 7.2. The reaction between the NHS ester end group of SMCC and the terminal amine group of PNIPAAm was allowed to proceed for 1 hour at room temperature with gentle mixing, generating a maleimide functionalized PNIPAAm. Fibronectin (50 uL, 1 mg/ml) was added to the reaction in which exposed thiol groups present in fibronectin readily reacted with sulfhydryl end groups present on SMCC functionalized PNIPAAm. The reaction was allowed to proceed at room temperature for 24 hours with gentle mixing. The products, PNIPAAm-maleimide-fibronectin, were freeze dried. The dried PNIPAAm-maleimide-fibronectin was then dissolved in PBS and mixed 1:1 (w/w) with type I bovine collagen. Electrostatic interactions between like-charged regions of collagen and fibronectin act to hold the blended materials together, thus generating a PNIPAAm-fibronectin-collagen material, PColFn that contains no collagen crosslinking.

### Phase Transition Characterization

### Differential Scanning Calorimetry (DSC)

Phase transition properties of amine terminated PNIPAAm, collagen, PCol, PColFn and UV PCol were analyzed by DSC (TA Instruments, 2910) using hermetic pans. Samples were dissolved to 15 mg/ml in de-ionized water and PBS and were stored at 4° C. prior to analysis. Thermal scans were performed at a rate of 5° C./min from 25 to 80° C.

### UV Spectrophotometry

A Cary 300. UV/VIS spectrophotometer was used to analyze the change in transmittance associated with the phase transition of PCol, UV PCol and PColFn scaffolds as they underwent a transformation from a relatively clear liquid to a milky white gel. The scaffolds were each dissolved in distilled water to a concentration of 1 mg/ml. Samples were placed in 4 ml UV cuvettes and were subjected to a heating rate of VC/min from 20 to 50° C. Transmittance measurements were taken every 0.5 min. To avoid bubble formation during heating, samples were sonicated briefly prior to testing.

### Gelling Time

The gelling time of the PCol scaffold was assessed as a function of temperature. A vial containing 5 mg/ml PCol was placed in a water bath at various temperatures and the time required for the sample to reach its cloud point was recorded.

### Gel Permeation Chromatography

The molecular weight (MW) of amine terminated PNIPAAm was determined by gel permeation chromatography using organic GPC. Both tetrahydrofuran (THF) and dimethylformamide (DMF) solvents were used in attempt to obtain the best approximation of the MW. Polystyrene and polyethylene oxide (PEO) standards were used for THF and DMF GPC respectively. Samples were compared with a commercially obtained PNIPAAm (Sigma) having a MW range of 20,000-25,000.

### Cell Culture

Human retinal pigment epithelial (RPE) cells were purchased commercially from ATCC and were cultured in CO₂ incubators (37° C., 5% CO₂, 95% air, 100% humidity). DMEM-F12 culture medium (Gibco) was supplemented with FBS (6.25% final concentration, Gibco), 1× glutamate (1% final concentration, Gibco), penicillin-streptomycin (1% final concentration, Gibco) and sodium bicarbonate (0.8% final concentration, Gibco). Calcein AM and ethidium homodimer (EthD-1) were purchased from Invitrogen and used to assess RPE viability in culture.

Several tests were performed to analyze the impact of the different scaffold components on retinal pigment epithelial viability in culture. The different tests included a cell supernatant assay in which scaffolds were added to pre-adhered cells, and a cell suspension assay in which cells were suspended within the scaffolds, thereby entrapping them within the gel upon heating. Scaffolds were pre-treated with a solution of PBS and penicillin-streptomycin (3:1 v/v) to remove biological contaminants. All tests were run in triplicate. Cell viability was assessed by staining cultured cells with calcein AM, EthD-1 and Hoescht.

### Cell Supernatant Assay

RPE cells were cultured in the presence of a variety of scaffolds including collagen, PNIPAAm, amine terminated PNIPAAm, PNIPAAm blended with collagen and PCol to assess scaffold cell compatibility. RPEs were seeded in a treated 48 well plate at a density of 10,000 cells per well. The plate was placed in a CO₂ incubator at 37° C. for 2 hours. Once the cells had adhered to the bottom of the wells, culture medium was removed, and replaced with 1 ml of DMEM-F12 containing 20 mg of dissolved scaffold. Following addition of the scaffold to pre-adhered cells, the culture dishes were returned to the 37° C. incubator, where the PNIPAAm-based scaffolds gelled in the supernatant of the plated cells. Media was changed after 48 hours and viability was assessed after 96 hours.

### Cell Suspension Assay

In the cell suspension assay, 100,000 RPEs were suspended in a solution of PCol (20 mg/ml PCol in DMEM-F12 medium). The suspension was added to a treated 48 well plate. The plate was then placed in a 37° C. CO2 incubator, where the PCol scaffolds gelled, entrapping the cells within their matrix. Culture media was changed every 2-3 days by collecting the supernatant and the gel in a 2 ml eppendorf tube, and centrifuging the mixture (2,500 RPM, 5 minutes, Eppindorf—Mini Spin Plus). Cells were stained at 4 and 14 days using calcein AM, EthD-1 and Hoechst. Images were captured with a Zeiss Axiovert inverted light microscope. Live and dead cell numbers were counted to assess viability.

Statistical analysis of means was done using Statistica 6.1 software (Statsoft Inc). Student's T-tests for independent samples were used to test for significance differences (p<0.05) between two groups, using Levene's test for homogeneity of variances.

### Environmental Scanning Electron Microscopy

High resolution images of the internal pore structure were obtained using an Electroscan 2020 environmental scanning electron microscope (ESEM). ESEM was used to examine unmodified PNIPAAm, PCol, UV PCol and PColFn. Scaffolds were swelled for 48 hours in distilled water at 37° C. The samples were rapidly frozen by immediate submersion in a liquid nitrogen bath to preserve the internal pore structure. The samples remained in the liquid nitrogen bath for 48 hours, at which time they were freeze dried for subsequent imaging. ESEM was also used to visualize RPE cells seeded within the PCol scaffold.

### Results and Discussion

### Synthesis of Amine Terminated PNIPAAm

Polymerization of amine terminated PNIPAAm yielded a flaky white powder that was soluble in aqueous solutions and demonstrated an LCST in the range of 32° C. Using both DMF and THF GPC having PEO and polystyrene standards respectively, amine terminated PNIPAAm was found to have a molecular weight in the range of 5,000 to 10,000.

### Synthesis of PNIPAAm-grafted-Collagen

### Synthesis of PCol via EDC/NHS Chemistry

Collagen crosslinking via EDC/NHS chemistry has been shown to be non-toxic both in vitro and in vivo. Therefore, this same chemistry was used to facilitate the grafting of amine terminated PNIPAAm chains to carboxylic acid groups of collagen. Having approximately 120 carboxylic acid groups per 1000 amine acid residues provides a high number of potential PNIPAAm grafting sites along the collagen backbone. By reacting collagen with an excess of PNIPAAm, it was possible to minimize collagen self-crosslinking, which can decrease the solubility of the material. Through EDC/NHS chemistry a cell friendly, comb-type graft polymer was synthesized that exhibits a phase transition below physiological temperatures, enabling the delivery of a liquid suspension of cells that forms a gel scaffold upon injection into the body. PCol and UV PCol are depicted in FIG. 9.

Following grafting, EDC, NHS and any un-grafted PNIPAAm chains were removed via extensive dialysis (MWCO 50,000), resulting in purified PCol that was freeze dried for subsequent use.

### Synthesis of UV PCol via Photocrosslinking

When exposed to UV light at 365 nm, riboflavin is excited into its triplet state leading to the generation of reactive oxygen species (ROS). The ROS react further, creating covalent linkages between primary amine and carboxylic groups. This reactivity has been used to drive the crosslinking of collagen. By introducing amine terminated PNIPAAm into a mixture of collagen, the ROS drive the formation of covalent linkages between amine groups of PNIPAAm and carboxylic acid groups of collagen, thus resulting in the grafting of PNIPAAm along the length of the collagen backbone. As a result of the non-specific generation of covalent linkages between amine and carboxyl groups, collagen crosslinking also results during this reaction. Significant collagen crosslinking results in a non-soluble gel. Solubility is essential for the ability to deliver cells as a liquid suspension thereby minimizing the invasiveness of cell transplantation. Therefore, to minimize collagen crosslinking, an excess of PNIPAAm was used to saturate potential reaction sites. The resulting UV PCol was a water soluble, temperature sensitive copolymer that undergoes a phase transition from liquid to gel below physiological temperature.

Fibronectin Functionalized PNIPAAm Combined with Collagen

Both previous methods for combining PNIPAAm and collagen resulted in collagen crosslinking to some extent. Although crosslinking has been minimized in the previous reaction schemes to give water soluble products, it was desirable to develop a PNIPAAm-collagen based scaffold that employed no collagen crosslinking. Simply blending PNIPAAm and collagen together would not be sufficient as the two materials would not stay together upon injection into the body. Furthermore, soluble collagen would likely leak into the vitreous chamber of the eye where it may cause complications. Therefore, a PNIPAAm-collagen based system was devised in which PNIPAAm was functionalized with fibronectin and then blended with collagen. Electrostatic interactions between like-charged regions of fibronectin and collagen act as a ‘glue’, holding the blended materials together. Thus, a rapidly gelling, temperature sensitive PNIPAAm-fibronectin-collagen copolymer was created in which no collagen crosslinking occurred.

SMCC, a heterobifunctional cross-linker that is frequently used in bioconjugate-chemistry, contains an amine reactive NHS ester end group. This allowed PNIPAAm to be coupled with SMCC, giving a maleimide functionalized PNIPAAm. The maleimide end group of SMCC specifically reacts with sulfhydryls, giving a sulfhydryl-reactive PNIPAAm that was used to couple with exposed thiol groups of fibronectin without the need for harsh reaction conditions. The result was a PNIPAAm-grafted-fibronectin copolymer. Although fibronectin contains cell adhesive RGD peptide sequences and could act as the bioactive component of the scaffold, it was decided to combine this PNIPAAm-grafted-fibronectin copolymer with collagen as collagen provides a superior 3-dimensional cell scaffold. Therefore, PNIPAAm-grafted-fibronectin was dissolved in PBS and combined with type I bovine collagen in a 1:1 (w/w) ratio to give a blend of PNIPAAm-grafted-fibronectin and collagen, PColFn.

### Phase Transition Characterization

DSC was used to examine the phase transitions of the different scaffolds. A heating rate of 5° C. per minute was used. Results are shown in Table 3.

TABLE 3

| Material | Transition Temperature (° C.) |
|---|---|
| PNIPAAm-NH₂ | 32 |
| Collagen | 54 |
| PCol | 32, 72 |
| PColFn | 36, 74 |
| UV PCol (1) | 31, 77 |
| UV PCol (2) | 31 |

The phase transition of PNIPAAm, 32° C., was conserved following functionalization with amine end groups. The denaturation of collagen, 54° C., was also observed using DSC. Multiple peaks were observed for the three synthesized scaffolds. The first peak is a function of phase transition from liquid to gel associated with the presence of PNIPAAm. The phase transition temperatures of the different scaffolds all occur below physiological temperatures. An increase in temperature, e.g. upon injection into the body, enables scaffold formation and subsequent cell entrapment. The secondary peaks were hypothesized to be a function of protein denaturation. To test the validity of this theory, UV PCol was subjected to a second round of heating in which the secondary transition temperature disappeared, indicating the secondary peaks were indeed present due to the denaturation of protein. Protein denaturation of the grafted biomaterials is significantly higher than the denaturation temperature of unmodified collagen, indicating that the modifications impart stability into the collagen.

### Turbidity Analysis

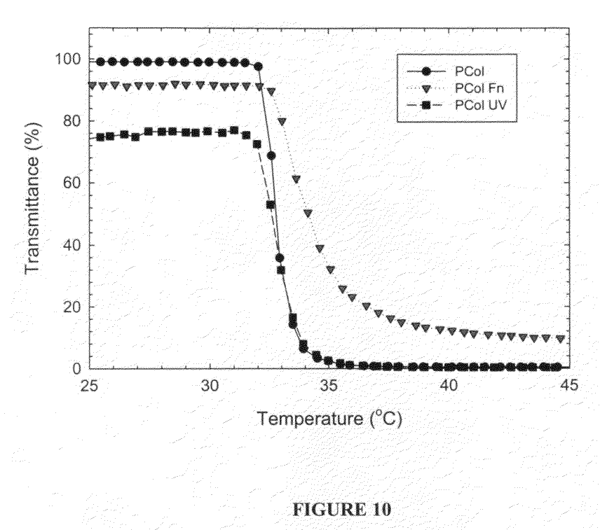

As the PNIPAAm-based scaffolds undergo a phase transition, they transform from a transparent liquid to a milky white, opaque gel. This change in clarity allows analysis of phase transition properties by analyzing transmittance of light through the sample as a function of temperature. The different scaffolds were dissolved in distilled water to a concentration of 1 mg/ml and percent transmittance was measured at temperatures ranging from 20 to 50° C. A heating rate of 1° C./min was used. Results are shown in FIG. 10.

The transition temperatures of the different scaffolds obtained via UV spectrophotometry correspond with previous DSC results. All three scaffolds appear to undergo rapid gelation at their LCST as indicated by the sudden decrease in transmittance rendering them suitable to deliver cells to a target site. Gelation times that are too slow allow leakage of the liquid scaffold and transplanted cells away from the site of injection. The goal is to isolate cells to the implantation site in order to maximize therapeutic efficiency as well as to minimize complications that may arise from leakage into the vitreous chamber of the eye.

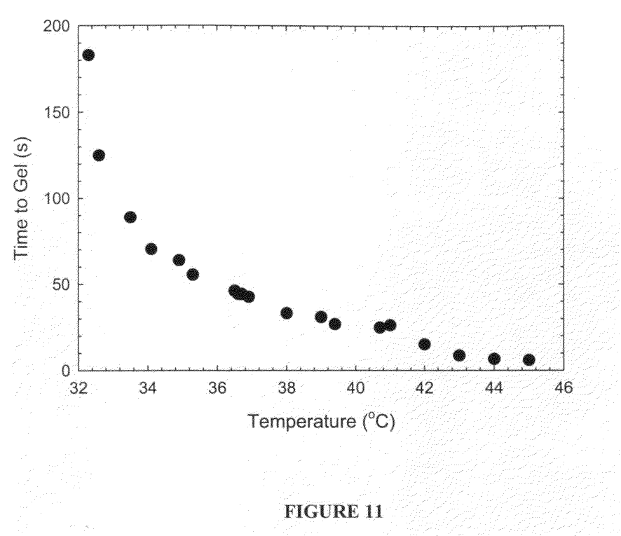

Gelling times of the PCol scaffold were analyzed by placing a vial (5 mg/ml PCol dissolved in distilled water) at room temperature into a water bath at temperatures varying from 25 to 45° C. and recording the time until cloud point was reached. The results are shown in FIG. 11. Rapid gelling times were observed and were found to decrease as a function of increasing temperature. At physiological temperature, the cloud point was reached in less than one minute. Although these measurements display rapid gelation, the results are limited by the heat transfer from the water bath to the contents of the vial. When PCol is injected directly into a water bath at temperatures above its LCST, gelation occurs almost instantaneously. Therefore, the PCol scaffold will gel rapidly upon injection into the body, entrapping cells and localizing treatment to the transplantation site.

### Environmental Scanning Electron Microscopy

ESEM images were collected for the different scaffolds and components in order to obtain some information about the 3-dimensional microenvironment within which transplanted cells would be entrapped. In order to view the internal structure of the hydrated materials, samples were swelled with distilled water for 2 days at 37° C. Samples were then rapidly frozen by immersion in a liquid nitrogen bath. After 2 days, water was removed via freeze-drying. The result was water-free scaffolds having their 3-dimensional pore structures preserved for imaging.

The ESEM images depict the differing microstructure obtained via different chemistries of synthesis. Unmodified PNIPAAm and PColFn display similarities with one another having ordered, interconnected porous networks. In contrast, PCol and UV PCol display highly amorphous, unordered microstructures with fibrillar strand-like structures likely resulting from collagen crosslinking. UV PCol has a higher degree of fibrils, which is consistent with a greater degree of collagen crosslinking. All scaffolds appear highly porous, which will allow diffusion of oxygen and nutrients into an out of the scaffolds providing nourishment for entrapped cells.

Images of RPE cells entrapped within the PCol scaffold were also obtained using ESEM. One million cells were loaded into a suspension of PCol (20 mg/ml) and prepared for imaging in the same fashion as previous samples. The RPE cells appear as circular dark spots within the PCol scaffold. It is interesting to note the degree of variability within the PCol scaffold itself; there are regions of highly organized, interwoven braid-like structures and regions of unordered globular structures. This variability likely results from differences in regional self-assembly of the copolymer.

### Cell Culture

### Cell Supernatant Assay

Following 96 hours of incubation with different scaffold components present in the culture medium, RPE cells were stained with calcein AM and EthD-1. Calcein stains live cells green and EthD-1 stains dead cells red. Cell viability of RPE cells was determined when seeded with a) culture medium, b) type I bovine collagen, c) unmodified PNIPAAm, d) a blend of unmodified PNIPAAm and collagen and e) PCol for 96 hours. Viabilities were all greater than 90% and there were no significant differences between the mean viabilities (T-test, p<0.05).

### Cell Suspension Testing

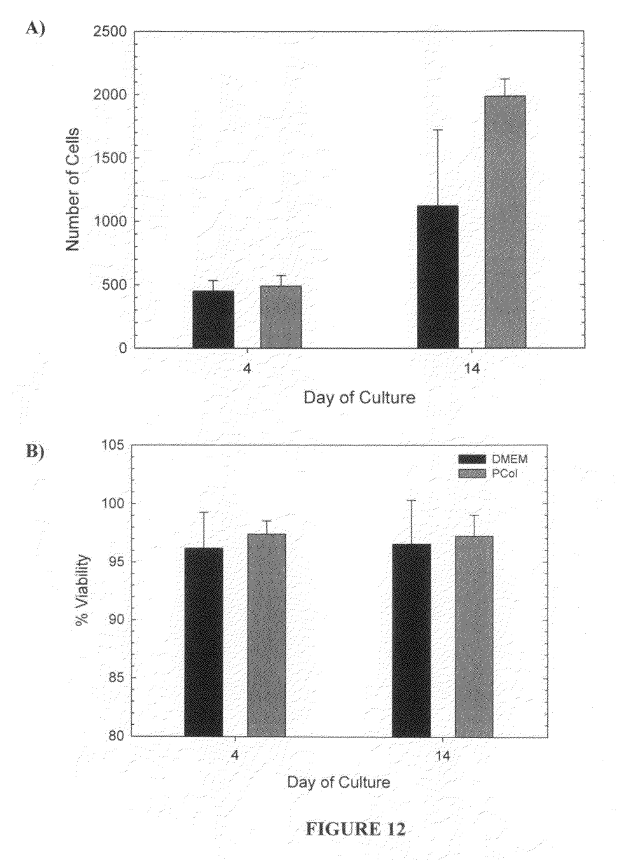

RPE cells were entrapped within a PCol suspension at densities of 10,000 and 100,000 cells per well and cultured for time periods of 4 and 14 days respectively. The cells were then stained with calcein AM, EthD-1 and Hoechst (FIG. 12A) to assess viability of the cells when cultured within a 3-dimensional PCol scaffold. RPE viability is also very high when seeded within the PCol matrix in a 3-dimensional scaffold. The cell count for the control conditions was higher than the PCol matrix at day 14. This was expected since the control, being a culture treated dish with DMEM F12 culture medium, represents ideal culture conditions. However, viability is over 95% for both the control and PCol scaffold at days 4 and 14 (FIG. 12B).

### CONCLUSIONS

Several cell friendly, temperature sensitive, in situ forming cell delivery scaffolds were synthesized. All three scaffolds consisted of a PNIPAAm component, which drives the gelation of the material from liquid to gel upon heating to body temperature. This phase transition will entrap transplanted cells within a scaffold, isolating the cells to the injection site in the subretinal space. This will ensure that when the cells eventually migrate out of the scaffold, they will remain in the subretinal space where integration into the damaged or diseased retinal tissue can occur. The collagen component of the scaffold creates a bioactive environment, promoting cells to undergo normal signalling pathways that will aid in their integration with host tissue and promote the release of neurotrophic factors.

While PCol and UV PCol both employ collagen crosslinking to some extent, this crosslinking has been minimized by saturating reaction sites with an excess of PNIPAAm, producing water soluble scaffolds that pass easily through a syringe. PColFn is another water soluble scaffold, but is completely free of collagen crosslinking. All three scaffolds exhibit a rapid phase transition from liquid to gel below physiological temperature, allowing the delivery of cells into the subretinal space as a liquid suspension that gels in situ. Furthermore, RPE cells demonstrate excellent viability when cultured in the presence of and within PCol, indicating that this scaffold is ideal for cell delivery purposes.

We claim:

1. A method of in situ hydrogel polymerization comprising the steps of:
1) modifying a biocompatible backbone polymer with an in situ polymerizable group to form a prepolymer solution;
2) administering the prepolymer solution to a target site; and
3) exposing the prepolymer solution to a stimulus that induces polymerization of the solution at the target site.

2. The method of claim 1, wherein the target site is an in vivo target site.

3. The method of claim 1, wherein the target site is an ophthalmic site.

4. The method of claim 1, wherein the stimulus is selected from the group consisting of heat and light.

5. The method of claim 4, wherein the stimulus is exposure to body temperature.

6. The method of claim 1, wherein at least one of a crosslinking agent, a facilitating agent, a stability agent, a polymerization initiating agent and a utility-specific component is added to the prepolymer solution.

7. The method of claim 6, wherein the polymerization initiating agent is a photo-initiator.

8. The method of claim 6, wherein the utility-specific component is a UV-absorbing molecule.

9. The method of claim 1, wherein the backbone polymer is selected from the group consisting of hyaluronate and collagen.

10. The method of claim 1, wherein the polymerizable group is selected from the group consisting of a methacrylate, an acrylamide, an acrylic acid, a urethane, silicone polymers and hydrogel polymers.

11. A prepolymer solution a comprising a collagen backbone and an acrylamide polymerizing agent.

12. The solution of claim 11, wherein the solution is polymerizable on exposure to body temperature.

13. A kit comprising a polymer backbone and a polymerizable group which may be combined to form a prepolymer solution that is polymerizable on exposure to body temperature.

14. The kit of claim 13, optionally comprising one or more of a crosslinking agent, a facilitating agent, a stability agent, a polymerization initiating agent and a utility-specific component.

15. A kit as defined in claim 13, wherein the polymer backbone and polymerizable group have been combined to form a prepolymer solution, and wherein the kit additionally comprises a polymerization initiating agent.

16. An article of manufacture comprising packaging material and a kit as defined in claim 13, wherein the packaging material is labeled to indicate that the prepolymer solution is for use to be administered to a target site for in situ polymerization on exposure to a stimulus that induces polymerization at the target site.

17. The article of claim 16, optionally comprising one or more of a crosslinking agent, a facilitating agent, a stability agent, a polymerization initiating agent and a utility-specific component.
